(19) United States
(12) Patent Application Publication
(10) Pub. No.: US 2009/0053817 A1
(43) Pub. Date: Feb. 26, 2009

(54) FIXED CHARGE REAGENTS

(75) Inventors: Gavin E. Reid, Victoria (AU); Kade D. Roberts, Victoria (AU); Henrik Neu, Uppsala (SE); Maria Liminga, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignees: LUDWIG INSTITUTE FOR CANCER RESEARCH, VICTORIA (AU); GE HEALTHCARE BIO-SCIENCES AB, UPPSALA (SE)

(21) Appl. No.: 11/573,362

(22) PCT Filed: Aug. 16, 2005
(86) PCT No.: PCT/SE2005/001214

§ 371 Date: Oct. 26, 2007

Related U.S. Application Data
(60) Provisional application No. 60/611,905, filed on Sep. 21, 2004.

(30) Foreign Application Priority Data
Aug. 16, 2004 (AU) 2004904613
Feb. 18, 2005 (SE) 0500415-5

(51) Int. Cl. G01N 33/00; C07C 233/01

(52) U.S. Cl. 436/86; 564/154

(57) ABSTRACT

The present invention relates to fixed charge reagents and kits for use in tandem mass spectrometry methods involving multiplex analysis. The compounds of the invention are phenacylamide compounds. The invention also relates to methods for the quantification of for example peptides and proteins by tandem mass spectrometry techniques using said reagents and kits. The reagents and kits of the invention enable multiplexed analysis of several samples in one experiment.

### CROSS REFERENCE TO RELATED APPLICATIONS

This application is a filing under 35 U.S.C. § 371 and claims priority to international patent application number PCT/SE2005/001214 filed Aug. 16, 2005, published on Feb. 23, 2006, as WO 2006/019354, which claims priority to patent application number 0500415-5 filed in Sweden on Feb. 18, 2005, and to application No. 60/611,905 filed in the United States on Sep. 21, 2004 and to application number 2004904613 filed in Australia on Aug. 16, 2004; the disclosures of which are incorporated herein by reference in their entireties.

### FIELD OF THE INVENTION

This invention relates to fixed charge reagents for use in tandem mass spectrometry methods that can be employed in for example proteome analysis. Especially, the present invention relates to modular stable isotope labelled fixed charge containing compounds that are useful as mass spectrometry (MS) reagents. It is also concerned with methods for the quantification of biomolecules, such as peptides and proteins by tandem mass spectrometry techniques using said reagents. The reagents of the invention enable multiplexed analysis of several samples in one experiment.

### BACKGROUND OF THE INVENTION

The goal of proteomics may be broadly defined as (i) the systematic identification of all the gene products i.e., proteins, expressed by a particular cell or tissue type at a given time, (ii) quantitative analysis of the differences in protein abundances observed between two different states of a biological system i.e., such as that encountered between a normal and diseased cell or tissue, (iii) identification and characterization of co- and post-translational protein modifications, and (iv) identification and characterization of the protein complexes and specific protein-protein interactions, involved in the regulation of cellular behaviour. It is widely anticipated that one of the major outcomes of proteomics research will be determination of the hitherto unknown functional role of the thousands of genes identified from recent genome sequencing initiatives, ultimately enabling a more complete understanding of the processes that control cellular biochemistry, as well as allowing the development of novel therapeutic agents targeted toward specific biomarkers of disease.

Substantial progress in the field has been achieved in recent years, primarily via developments in the application of mass spectrometry (MS) and associated sample handling methodologies for protein and peptide purification, separation and analysis, along with sophisticated bioinformatic tools for rapid protein identification and characterization via database interrogation of MS derived data. However, there are a number of issues that currently limit the ability of these approaches, such as the enormous dynamic range and mixture complexity associated with the proteome. Furthermore, most conventional methods for protein identification and characterization are based on MS analysis of individual peptides obtained following proteolytic digestion of an individual protein of protein mixture of interest, thereby further increasing the mixture complexity problem.

Peptide mixture complexity presents a particular problem for the quantitative analysis of protein abundances. To date, the majority of methods for protein quantification have employed either in vivo or in vitro labelling using naturally abundant or isotopically enriched derivatives, to introduce a differential mass ‘tag’ between two different samples of interest (e.g. a normal verus diseased cell or tissue state). By comparing the relative abundances of the peptide ions obtained from an isotopically enriched sample with those from a sample prepared using naturally abundant isotopes, quantification of differences in protein abundance between the two samples may be obtained. These methods have also been applied to the quantitative analysis of phosphorylation status within post translationally modified proteins by β-elimination of phosphoserine and phosphothreonine residues under strongly alkaline conditions to yield dehydroalanine or dehydroaminobutyric acid residues, respectively followed by Michael addition of naturally abundant or isotopically enriched nucleophiles.

A range of both ‘targeted’ and ‘global’ labelling strategies for protein quantification have been described. The majority of these methods however, have been based on a common approach for identification of the differential mass “signature” between the two samples i.e., by mass analysis of their intact peptide precursor ions. Thus, limitations are encountered when; (i) when one or both of the differentially labelled ions of interest are present at low levels (for example, approaching or below the limit of detection of the mass spectrometer), (ii) the m/z values of low abundance differentially labelled peptide ions overlap with other higher abundance components present in the mixture, (iii) separation of the differential labelled “heavy” and “light” peptides occurs during chromatographic fractionation of the peptide mixture or (iv) the mass spectrometer lacks sufficient resolution to adequately resolve the two labelled components, thereby precluding their detection.

MS/MS methods have also been extensively employed previously for the identification of peptides containing selected structural features via formation of diagnostic “signature” product ions or characteristic product ions from cross linked peptides, via neutral loss and/or precursor ion scan modes of analysis. These methods have been demonstrated to yield greater specificity and increase sensitivity by 1-2 orders of magnitude over conventional MS based detection methods, due to the reduction in chemical noise associated with the formation of product ions at different m/z values to that of the mass selected precursor ion. However, similar issues to those mentioned above limit the general applicability of these methods.

Recently, an MS/MS based approach for selective protein identification and quantification was described in WO 04/046731, the disclosure of which is incorporated herein by reference. In contrast to all of the MS and MS/MS based derivatization strategies outlined above, this approach is based on the formation of ‘fixed-charge’ derivatives on the side-chains of selected amino acids, or on the side-chains of selected amino acid residues contained within a protein or peptide. These side-chain fixed-charge derivatives are designed to direct the dissociation of the amino acid, peptide or protein ion containing the fixed charge toward exclusive formation of a single product ion that is characteristic of fragmentation occurring at the site of the fixed-charge, thereby allowing its selective identification by neutral loss scan mode MS/MS methods. By incorporation of ‘light’ and ‘heavy’ isotopically encoded labels into the fixed-charge derivatives, these methods have also been extended to the quantitative analysis of differential protein expression with significantly improved sensitivity and selectivity.

However, a limitation of this fixed charge derivatization approach for quantitative protein analysis, is that two separate MS/MS scans must be acquired in order to determine abundance ratios, i.e., one MS/MS scan for ‘light’ labelled peptide ions, followed by an MS/MS scan for ‘heavy’ labelled peptide ions.

Simultaneous quantitative analysis of protein abundance in more than two samples has recently been described. The samples of interest are selected based on a common property and then, in a second step, the samples are separated by altering the common property. The analysis of the separated samples is performed in a single step in the same MS/MS scan and is called a multiplexed analysis.

WO 01/68664 describes tandem mass tags in the form of a set of two or more mass labels, each label in the set comprising a mass marker moiety attached via a cleavable linker to a mass normalisation moiety. The aggregate mass of each label in the set may be the same of different and the mass of the mass marker moiety of each label in the set may be the same or different. The mass labels are isobaric and enable high degree of multiplexing.

WO 02/014867 relates to a set of reporter signal peptides having a common property allowing the reporter signal peptides to be separated from molecules lacking the common property. The reporter signal peptides have the ability of being altered in such a way that they can be distinguished from every other altered form of reporter signal peptide. Thus, this document also relates to isobaric mass labels with multiplexing capability.

WO 04/070353 relates to an analysis method using isobaric mass labels. The labelling reagents comprise a reactive group, a bond, a linker, a further bond and a reporter moiety. The two bonds fragment in at least a portion of the labelled analytes when subjected to dissociative energy. The mass labels enable multiplexing of more than two samples simultaneously.

In spite of the disclosures in the above documents, there is still a need for improved reagents for multiplexed MS analysis.

### SUMMARY OF THE INVENTION

The present invention relates to novel ‘modular’ isotopically labelled fixed charge derivatization reagents, and tandem mass spectrometry methods to enable the ‘multiplexed’ quantitative analysis of biomolecules, such as amino acids, peptides and proteins in a single MS/MS experiment.

The present invention relates to novel fixed-charge derivatization reagents. The reagents may be selective for reaction with the N-terminal amino groups of amino acids, peptides and proteins, or with amino containing side chains of amino acids such as lysine, or with guanidine containing side chains of amino acids such as arginine or homoarginine, or with thiol containing side chains of amino acids such as cysteine or homocysteine, or with indole containing side chains of amino acids such as tryptophan, or with dehydroalanine or dehydroamino-2-butyric acid amino acids formed by β-elimination from O-linked phosphorylated tyrosine or glycosylated serine or threonine, or with O-linked phosphorylated serine or threonine, or with peptides or proteins comprising at least one residue of such amino acids. These novel reagents have potential application for the high throughput, sensitive and selective quantification of these compounds when present in complex mixtures.

Accordingly, the present invention provides fixed charge reagents of formula XM₁M₂⁺ or salts thereof, wherein:

X is a reactive group specific to a functional group contained within biomolecule, such as an amino acid, peptides or proteins, or peptides or proteins containing at least one of such amino acid.
M₁ is a first module selected from branched alkyl optionally interrupted or substituted with an alkyl, aryl, substituted alkyl, substituted aryl, amino, amide, acid, ester or thioester, and is optionally isotopically encoded by incorporation of one or more of ²H, ¹³C, ¹⁵N or ¹⁸O;
M₂⁺ is a second module group containing a fixed charge and is selected from the group consisting of a tertiary alkyl or aryl sulfonium ion, a quaternary alkyl or aryl ammonium ion or a quaternary alkyl or aryl phosphonium ion, and is optionally isotopically encoded by incorporation of one or more of ²H, ¹³C, ¹⁵N or ¹⁸O.

Thus, in a first aspect the present invention provides a compound of formula XM₁M₂⁺, or a salt thereof,

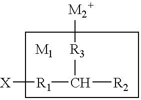

wherein,
X is a biomolecule reactive group, such as a thiol, a thiol reactive group, an amino reactive group, a guanidino reactive group, or a reactive group specific for the C₂-indole position of the side chain of Trp or a phosphate reactive group.

When X is a thiol, such as —SH, the present invention provides a compound of formula XM₁M₂⁺, or a salt thereof, for specific reaction with dehydroalanine or dehydroamino-2-butyric acid formed by β-elimination from O-linked phosphorylated or glycosylated serine or threonine, or dehydroalanine or dehydroamino-2-butyric acid residues formed by β-elimination from O-linked phosphorylated or glycosylated serine or threonine containing proteins or peptides respectively.

The thiol reactive X group may be selected from the group consisting of a halide, a disulfide exchange group, a vinyl group or a N-methyl maleimide. The halide is preferably —Cl, —Br, or —I. The disulfide exchange group may be selected from —S—S—R′ where R′ is —C₆H₅, 3-carboxyl-4-nitrophenyl, 2,4-dinitrophenyl, 4-nitrophenyl, 2-nitrophenyl, 2-pyridyl, 5-nitropyridyl, 3-nitropyridyl, methanesulfonyl. Where X is a vinyl group it may be —CH═CH₂.

The amino reactive X group, i.e. reactive with peptide N-terminals or lysines, may be selected from the group consisting of an acid anhydride, an active ester, such as an NHS (N-hydroxysuccinimide)-ester, an acid halide, a sulfonylhalide, a substituted O-methyl isourea, an isocyanate or an isothiocyanante.

The guanidino reactive X group may be selected from the group consisting of a substituted 2,3-butanedione, a substituted 2,4-pentanedione, a substituted glyoxal, or a substituted phenylglyoxal.

The reactive X group specific to the C2-indole position of the side chain of tryptophan or tryptophan containing proteins or peptides may be selected from a halide or a dimethyl sulfonium ion. The halide is preferably —Cl, —Br, or —I. The dimethyl sulfonium ion is —S(CH₃)₂⁺. When X is the foregoing, R₁, see below, is selected from a -2-hydroxy-5-nitrobenzyl-, or -(2-hydroxy-5-nitrobenzyl)-4-Y— group.

When X is a reactive group specific to the C2-indole position of the side chain of tryptophan it may also be selected from a sulfenylhalide. The sulfenylhalide is preferably —SCl, —SBr, or —Si. In this case, R₁, see below, is selected from a -2-nitrophenyl- or -(2-nitrophenyl)-4-Y— group.

The O-phosphate reactive X group may be selected from imidazole and diazoalkane.

M₁ is —R₁CH(R₃)R₂, where R₁ is selected from —(CH₂)ₙ—, —Y— or —(CH₂)ₙY— or is absent; R₂ is —YCH₂COC₆H₅ or, when X is a phosphate reactive group, R₂ is selected from —(CH₂)ₙH, —C₆H₅, —CH₂C₆H₅, —NH₂, —YH, —Y(CH₂)ₙH, —YC₆H₅, —YCH₂C₆H₅; and R₃ is —(CH₂)ₙ—, and is optionally isotopically encoded by incorporation of one or more of ²H, ¹³C, ¹⁵N or ¹⁸O in which case M₁ is referred to as M₁′; n is from 1 to 3 inclusive; Y is selected from CONH, NHCO and COO; and M₂⁺ is attached to the R₃ group of M₁ and is selected from the group consisting of a tertiary alkyl or aryl sulfonium ion, —S⁺CH₃R″, where R″ is selected from —CH₂COC₆H₅, —CH₂COC₆H₅CH₃, and —CH₂COC₆H₅CH₂CH₃, or a quaternary alkyl or aryl ammonium ion —N⁺(R′″)₃, or a quaternary alkyl or aryl phosphonium ion —P⁺(R′″)₃, where R′″₃ is —(CH₂)ₙH (where n=1 to 3), —C₆H₅, —CH₂C₆H₅, —CH₂COC₆H₅ and wherein M₂+ optionally is isotopically encoded by incorporation of one or more ²H, ¹³C, ¹⁵N or ¹⁸O and is in that case referred to as M₂′⁺.

In a preferred embodiment, M₁ is —R₁CH(R₃)R₂, where R₁ is —(CH₂)ₙY—, wherein Y is CONH; R₂ is —YCH₂COC₆H₅; and R₃ is —(CH₂)ₙ—, and M₂⁺ is —S⁺CH₃R″, wherein R″ is —CH₂COC₆H₅, and M₁ and/or M₂⁺ is/are optionally isotopically encoded by incorporation of one or more of ²H, ¹³C, ¹⁵N or ¹⁸O. n=1 or 2.

A preferred fixed charge reagent according to invention comprises the following structure

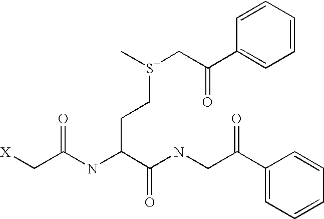

X may be any reactive group reactive with any biomolecule, such as a native or modified protein, peptide, nucleic acid or carbohydrate, preferably a protein/peptide reactive group as defined above. Preferably, X is selected from a thiol reactive group, an amino reactive group or a phosphate reactive group. Examples are provided above.

For Cys-labelling the most preferred X-groups are Br or I. For amino labelling the most preferred group is an NHS-ester.

In a second aspect, the present invention provides a reagent kit for quantitative analysis of amino acids, peptides or proteins by tandem mass spectrometry, comprising a container comprising one or more fixed charge reagents of the formulae XM₁′M₂⁺, XM₁M₂′⁺ and XM₁′M₂′⁺.

In another aspect, the present invention also extends to compounds consisting of amino acids, peptides or proteins, that have been derivatized with a compound of formulae XM₁′M₂⁺, XM₁M₂′⁺ and XM₁′M₂′⁺ as defined above.

In another aspect, the present invention provides a reagent kit comprising a container containing compounds consisting of amino acids, peptides or proteins that have been derivatized with a compound of formulae XM₁′M₂⁺, XM₁M₂′⁺ or XM₁′M₂′⁺ as defined above.

The kits may also include instructions for use of the compounds of the invention in the quantitative analysis of amino acids, peptides or proteins by mass spectrometry.

The reagent kits further may also contain one or more containers containing: cysteine disulfide reducing agents, alkylating agents, proteases or chemical cleavage agents, and/or solvents. The cysteine disulfide reducing agents preferably include dithiothreitol (DTT), mercaptoethanol, tris-carboxyethyl phosphine (TCEP), and/or tributylphosphine (TBP). The cysteine alkylating agents preferably include alkylhalides (e.g. iodoacetic acid, iodoacetamide), vinylpyridine or acrylamide. The proteases or chemical cleavage agents preferably include trypsin, Endoproteinase Lys-C, Endoproteinase Asp-N, Endoproteinase Glu-C, pepsin, papain, thermolysin, cyanogen bromide, hydroxylamine hydrochloride, 2-[2′-nitrophenylsulfenyl]-3-methyl-3′-bromoindole (BNPS-skatole), iodosobenzoic acid, pentafluoropropionic acid and/or dilute hydrochloric acid. The solvents preferably include urea, guanidine hydrochloride, acetonitrile, methanol and/or water.

Preferred reagent kits comprise one or more of the following reagents.

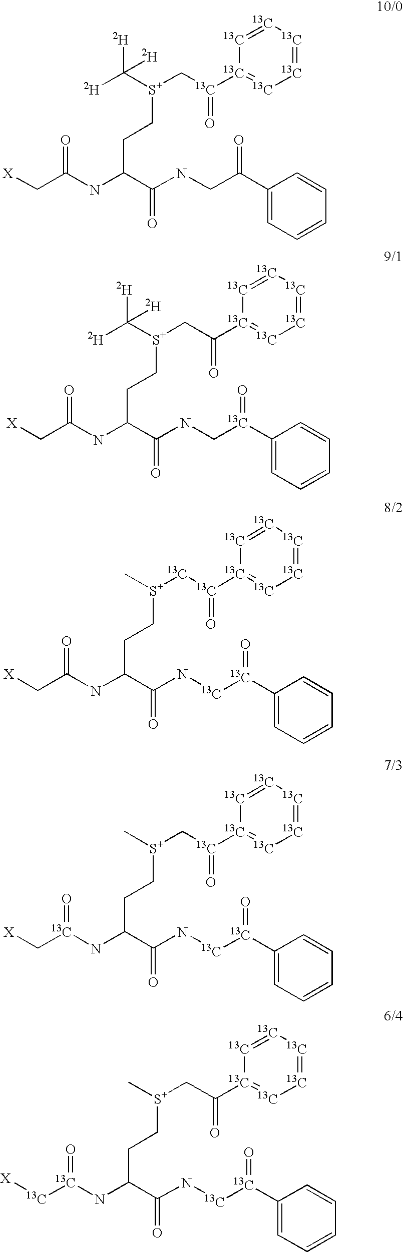

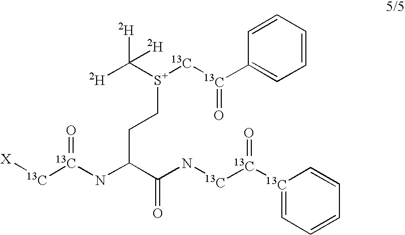

In the above reagent 5/5, the ¹³C-label can be at any position in the lower ring. The shown formula is only shown in illustrative purpose.

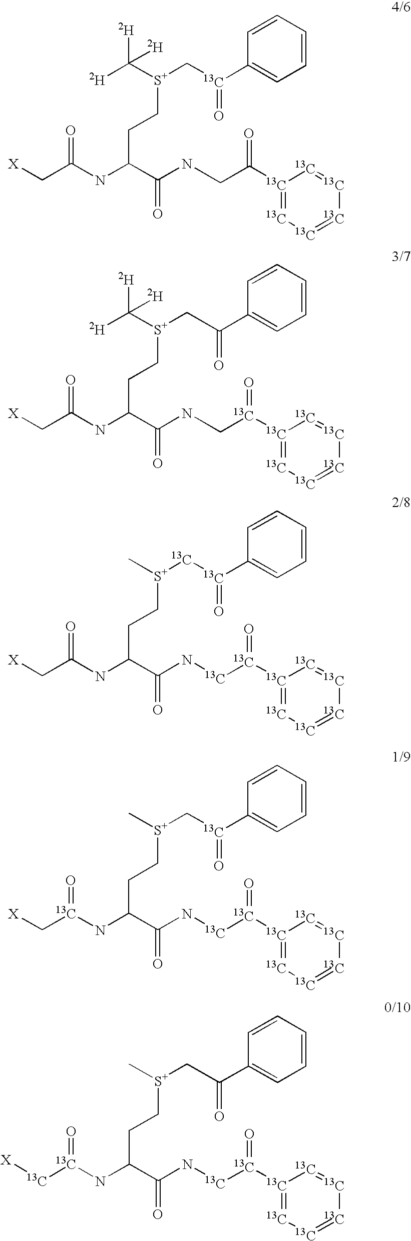

For 6-plex analyses, the above reagents 10/0, 8/2, 6/4, 4/6, 2/8 and 0/10 may be used. For 2-plex analyses, preferably 10/0 and 0/10 is used.

Alternatively, the kit comprises the following preferred reagents:

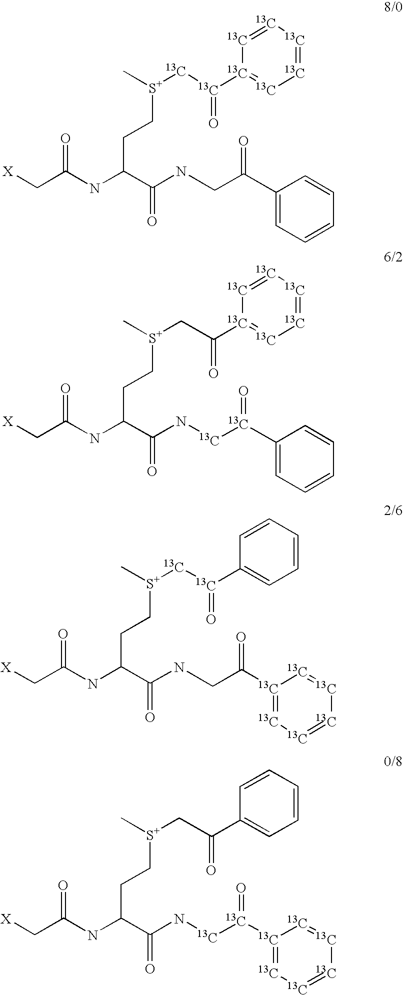

If only a 2-plex analysis is required, then preferably the above reagents 8/0 and 0/8 are used.

In both preferred sets of reagents above, X is any protein reactive group as defined above and is preferably a thiol reactive group selected from Br or I, preferably Br.

The positioning of the isotopes within M₁ and M₂⁺ is not critical for the invention and may vary as long as the desired mass difference is obtained.

Use of the set of eleven reagents enables multiplexing of eleven samples in one experiment. The mass difference between the labels is 1-10 Da.

In each reagent, M₁ and M₂⁺ are mass balanced against each other.

In MS/MS, M₂⁺ is fragmented off but the mass difference in M₁ remains which means that the eleven samples can be separated from each other. The mass differences are, as appears in the above formulas: 0, 1, 2, 3, 4, 5, 6, 7, 8, 9 and 10 Da from the first to the last of the eleven reagents. A kit according to the invention comprises two or more of the above reagents.

Use of the set of four reagents enables multiplexing of four samples in one experiment. The mass difference between the labels is 2, 4, 6 or 8 Da.

In each reagent, M₁ and M₂⁺ are mass balanced against each other. In MS/MS, M₂⁺ is fragmented off but the mass difference in M₁ remains which means that the four samples can be separated from each other. The mass differences are, as appears in the above formulas: 0, 2, 6 and 8 Da from the first to the last of the four reagents. A kit according to the invention comprises two or more of the above reagents.

The reagents according to the invention may be used as research tools and are especially suited for proteomic research. Moreover, the reagents may be used in medicine to predict, detect and diagnose different conditions in humans and animals which are related to protein or peptide changes in humans or animals.

The invention in another aspect provides methods for providing an internal standard in a mass spectrometer method comprising adding to a sample a predetermined quantity of an isotopically encoded fixed charge derivatized amino acid, peptide or protein described above.

The fixed charge reagents of the present invention are suitable for use in the tandem mass spectrometry methods for quantitative analysis described below. In this embodiment, the reagents of the invention are employed for the fixed-charge derivatization of an amino acid, peptide or protein, to enable their quantification via selective and directed fragmentation during MS/MS dissociation.

Thus, in a third aspect, the present invention provides a method for quantitative analysis of biomolecules such as amino acids, peptides or proteins, using the above reagents and/or kits, the method comprising:
(1) providing a mixture of amino acids, peptides or proteins containing at least one selected amino acid, peptide or protein, or peptide or protein comprising at least one residue of the selected amino acid, derivatized to contain a fixed-charge using compounds of formula XM₁′M₂⁺, XM₁M₂′⁺ and XM₁′M₂′⁺, or salts thereof, as described above; the peptides in the sample mixture are preferably separated by liquid chromatography, preferably cation chromatography, to separate the positive charge labelled peptides from unlabelled peptides;
(2) passing the mixture of amino acids, peptides or proteins containing at least one derivatized amino acid or derivatized amino acid residue containing peptide or protein, through a first mass resolving spectrometer to select precursor protein or peptide ions having a first mass-to-charge ratio;
(3) subjecting the precursor ions of the first mass-to-charge ratio to dissociation to form a product ion having a second mass-to-charge ratio that is characteristic of the loss of M₂ or M₂′ at the site of the fixed-charge; and
(4) detecting the product ions having the second mass-to-charge ratio.

The method of analysis may be used for the identification and/or quantification of amino acids, peptides or proteins.

Preferably the amino acid, peptide or protein contains an N-terminal amino group, a cysteine, a homocysteine, a lysine, an arginine, a homoarginine, a tryptophan, a dehydroalanine or a dehydroamino-2-butyric acid or a phosphate group.

The method of the latter aspect of the invention may include the preceding step of derivatizing the amino acid, peptide or protein with a compound of formula XM₁′M₂⁺, XM₁M₂′⁺ and XM₁′M₂′⁺, or salts thereof.

Preferably, the method of the invention described above comprises the further step of: (5) determining the identity of the peptide or protein.

Step (5) may be performed by first repeating steps (1), (2), and (3) and then subjecting the product ion having the second characteristic mass-to-charge ratio formed by loss from the precursor to a further stage of dissociation to form a series of product ions having a range of mass to charge ratios, for the purpose of determining the amino acid sequence of the peptide or protein and subsequently, the identity of the protein of origin.

Alternatively, step (5) may be carried out by use of high resolution mass analyzers to obtain an “accurate mass tag” (i.e., a mass accuracy of, for example, approximately 1-5 ppm) on the product ion detected in step (4). This, coupled with database searching, may be employed for subsequent identification of those peptides found to contain a fixed-charge derivative.

The amino acid, peptide or protein ion may be dissociated by any suitable dissociation method including, but not limited to, collisions with an inert gas (known as collision-induced dissociation (CID or collisionally-activated dissociation (CAD); (ii) collisions with a surface (known as surface-induced dissociation or SID); (iii) interaction with photons (e.g. via a laser) resulting in photodissociation; (iv) thermal/black body infrared radiative dissociation (BIRD), (v) interaction with an electron beam, resulting in electron-induced dissociation for singly charged cations (EID), electron-capture dissociation (ECD) for multiply charged cations, or combinations thereof, or (vi) by electron transfer dissociation (ETD).

Analysis of the amino acid, peptide or protein ion may be performed by tandem mass spectrometry. The tandem mass spectrometer may be equipped with electrospray ionization (ESI) or matrix assisted laser desorption ionization (MALDI) interfaces to transfer the protein or peptide ion from solution into the gas-phase.

The methods of the invention in certain embodiments may also include one or more steps of protein extraction, protein separation, reduction and alkylation of cysteine disulfides and/or protein digestion.

### BRIEF DESCRIPTION OF THE DRAWINGS

FIG. 1 is the schematic representation of the use of the reagents of the present invention for the ‘multiplexed’ quantification of protein abundances observed between different samples in a single product ion scan mode MS/MS experiment.

FIGS. 2a, 2b and 2c are the schematic representations of the use of six reagents of the present invention for the ‘multiplexed’ quantification of protein abundances observed between different samples in a single product ion scan mode MS/MS experiment.

### DEFINITIONS

“Fixed-charge”, as used herein, includes any charge localised to a specific heteroatom contained within a specific heteroatom contained within the derivatization reagent, by the attachment of any moiety.

“Fixed-charge derivatization”, as used here, means the introduction of a fixed-charge as defined above.

“Protein”, as used herein, means any protein, including, but not limited to peptides, enzymes, glycoproteins, hormones, receptors, antigens, antibodies, growth factors, etc., without limitation. Proteins may be endogenous, or produced from other proteins by chemical or proteolytic cleavage. Preferred proteins include those comprised of at least 15-20 amino acid residues.

“Peptide” as used herein includes any substance comprising two or more amino acids and includes di-, tri-, oligo and polypeptides etc according to the number of amino acids linked by amide (s) bonds. Peptides may be endogenous, or produced from other peptides or proteins by chemical or proteolytic cleavage. Preferred peptides include those comprised of up to 15-20 amino acid residues.

When the amino acids are α-amino acids, either the L-optical isomer or the D-optical isomer can be used. The L-isomers are generally preferred. For a general review, see, [Spatola, A. F., in Chemistry and Biochemistry of amino acids, peptides and proteins. 1983, B. Weinstein, eds., Marcel Dekker, New York, p. 267.]

The term “salt thereof” includes any suitable counter ion. Non-limiting examples of counter ions are halide ions such as chloride, bromide, iodide and acetate, trifluoroacetate, tetrafluoroborate.

“Quantitative analysis” means absolute quantification, relative quantification, analysis in the purpose of detection, diagnose, etc

### DETAILED DESCRIPTION OF THE INVENTION

The present invention will now be described with reference to particular embodiments, however, the method of the present invention is not to be considered limited to these particular embodiments.

### Synthesis of Fixed Charge Reagents

First a general synthesis of preferred reagents of the invention will be described. Thereafter the specific synthesis of one preferred fixed charge reagent will be described.

### EXAMPLES

The present examples are provided for illustrative purposes only, and should not be interpreted in any way as limiting the scope of the invention as defined by the appended claims. All references provided below and elsewhere in the present specification are hereby included herein via reference.

### Example 1

### Preparation of [3-(2-X-acetylamino)-3-(2-oxo-2-Phenyl-ethylcarbamoyl)-propyl]-methyl-(2-oxo-2-phenyl-ethyl)sulfonium salt (X-Met-diAP) (X═Br, I, C═C, CH₂-malemide, SS-phenyl)

A non-limiting method for the preparation of [3-(2-X-acetylamino)-3-(2-oxo-2-phenyl-ethylcarbamoyl)-propyl]-methyl-(2-oxo-2-phenyl-ethyl)sulfonium salt (X-Met-diAP) 6 was achieved by using methods known to those skilled in the art, via addition of phenacylamine (1.1 eq) 2 to Boc-L-methionine hydroxysuccinimide (Boc-Met-OSu) (1.1 eq) 1 dissolved in tetrahydrofuran and triethylamine (2.5 eq). Tetrahydrofuran was removed under vacuum, the crude dissolved in dichloromethane, washed with NaHCO₃ (sat), dried and concentrated under vacuum to yield 3 as an amber oil, which crystallize upon standing.

In equal amounts of dichloromethane and trifluoro acetic acid the oil 3 was dissolved and left until free amine 4 was obtained. After concentration the corresponding acid halide and diisopropylethylamine or triethylamine was added. After workup and solvent removal X-Met-Ap was obtained and purified by RP semi-preparative HPLC. X-Met-Ap (1 eq) 5 and phenacylbromide (2-5 eq) was dissolved in acetonitril, water and acetic acid and allowed to react for 20 hours. After evaporation the final product 6 was purified by RP semi-preparative HPLC and collected fractions were lyophilized to yield the final product as a white salt.

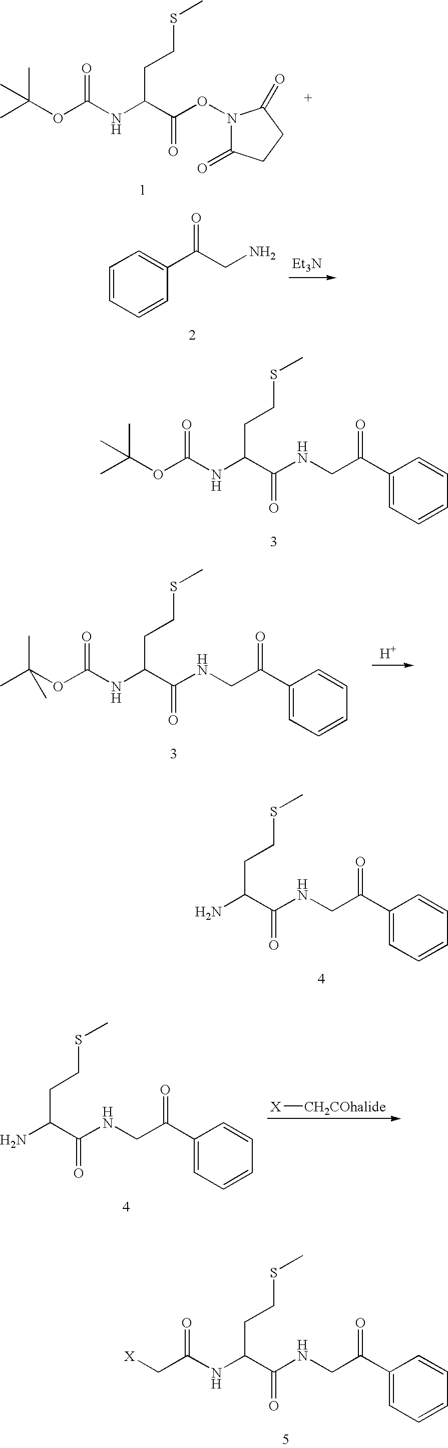

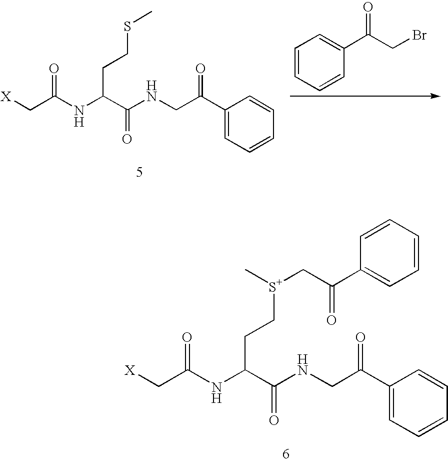

### Example 2

### Preparation of [3-(2-bromo-acetylamino)-3-(2-oxo-2-phenyl-ethylcarbamoyl)-propyl]-methyl-(2-oxo-2-phenyl-ethyl)sulfonium bromide (Br-Met-diAP)

A non-limiting method for the preparation of [3-(2-bromo-acetylamino)-3-(2-oxo-2-phenyl-ethylcarbamoyl)-propyl]-methyl-(2-oxo-2-phenyl-ethyl)sulfonium bromide (Br-Met-diAP) 6 was achieved by using methods known to those skilled in the art, via addition of phenacylamine (1.1 eq) 2 to Boc-L-methionine hydroxysuccinimide (Boc-Met-OSu) (1.1 eq) 1 dissolved in tetrahydro furan and triethylamine (2.5 eq). Tetrahydro furan was removed under vacuum, the crude dissolved in dichloromethane, washed with NaHCO₃ (sat), dried and concentrated under vacuum to yield 3 as an amber oil.

In equal amounts of dichloromethane and trifluoro acetic acid the oil 3 was dissolved and left until free amine 4 was obtained. After concentration, water was added and pH adjusted to 8-9 by addition of NaHCO₃ (sat) followed by addition of bromoacetyl bromide (2 eq) dissolved in dichloromethane. The phases were separated and the water phase extracted using dichloromethane, the combined organic phases were dried and solvent removed under vacuum to yield 5. To the purified (RP semi-preparative HPLC) Br-Met-Ap (1 eq) 5, phenacylbromide (2-5 eq) was dissolved in acetonitril and allowed to react for 20 hours. After evaporation the final product 6 was purified by RP semi-preparative HPLC and collected fractions were lyophilized to yield the final product as a white salt.

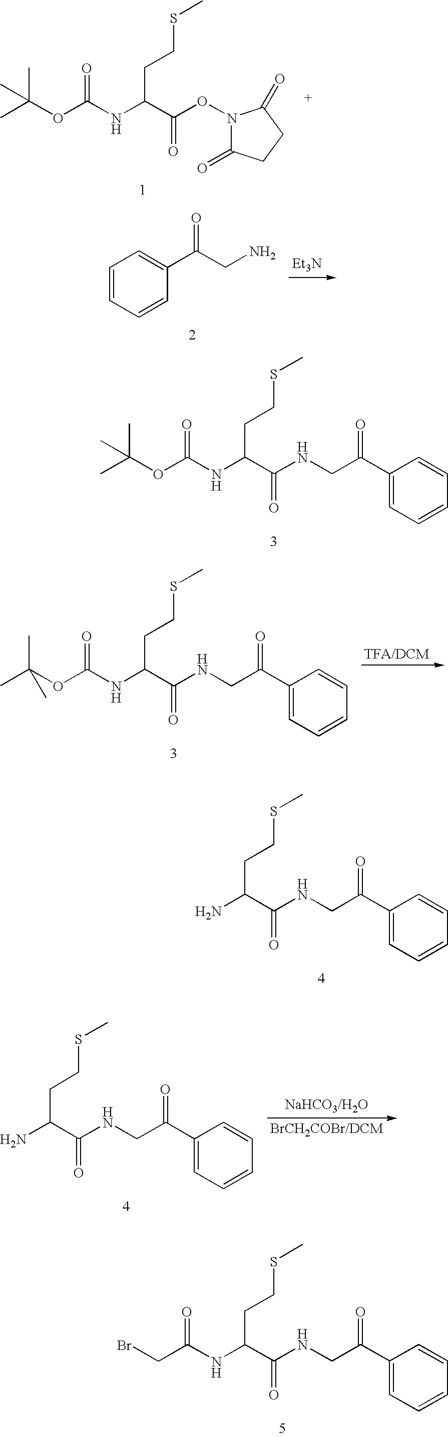

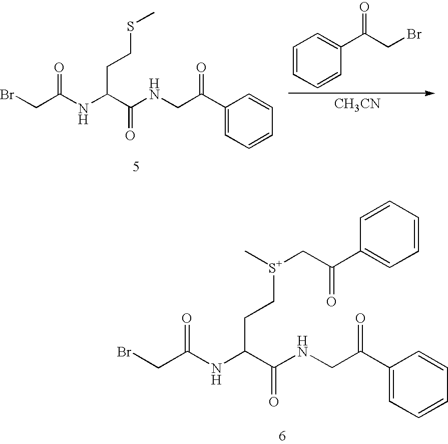

### Labelled Reagents

¹³C₈-, ¹³C₇-, ¹³C₆-, ¹³C₂-, ¹³C₁-Phenacylbromide was prepared via Friedel-Craft reaction using ¹³C₆- or ¹³C₁-benzene or benzene and ¹³C₂- or ¹³C₁-bromoacetyl bromide or bromoacetyl bromide. ¹³C₂- and ¹³C₁-bromoacetyl bromide was prepared from ¹³C₂- and ¹³C₁-bromoacetic acid and oxalylbromide. The corresponding ¹³C-labelled phenacylamine was prepared via amination using the corresponding ¹³C-labelled phenacylbromide and hexamethylenetetramine followed by HCl cleavage. Deuterium labelled Boc-(D₃)Met-OSu was prepared via Boc-protection of D₃-Met and NHS ester formation.

The labelled reagents (Br-Met-diAp) were then prepared via the synthesis procedure described above using the labelled intermediates where appropriate.

### Multiplex Analysis

The present invention enables multiplex analysis of several samples in one experiment. As described in example 4, six samples are analysed simultaneously.

### Example 3

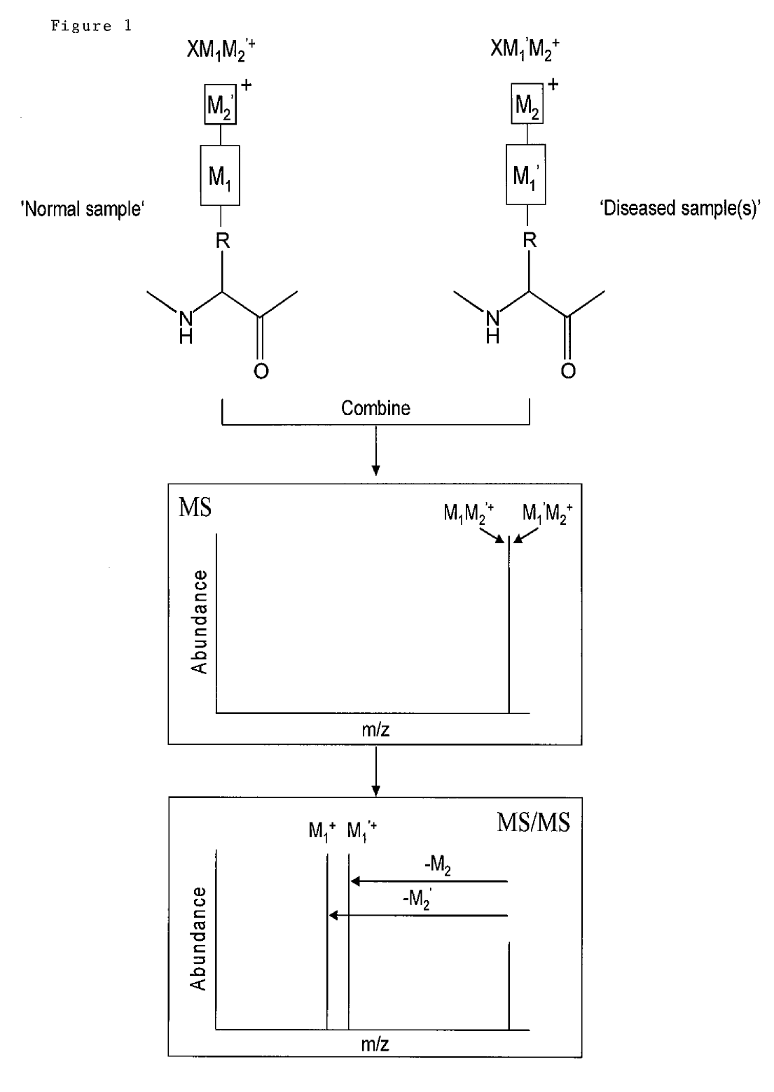

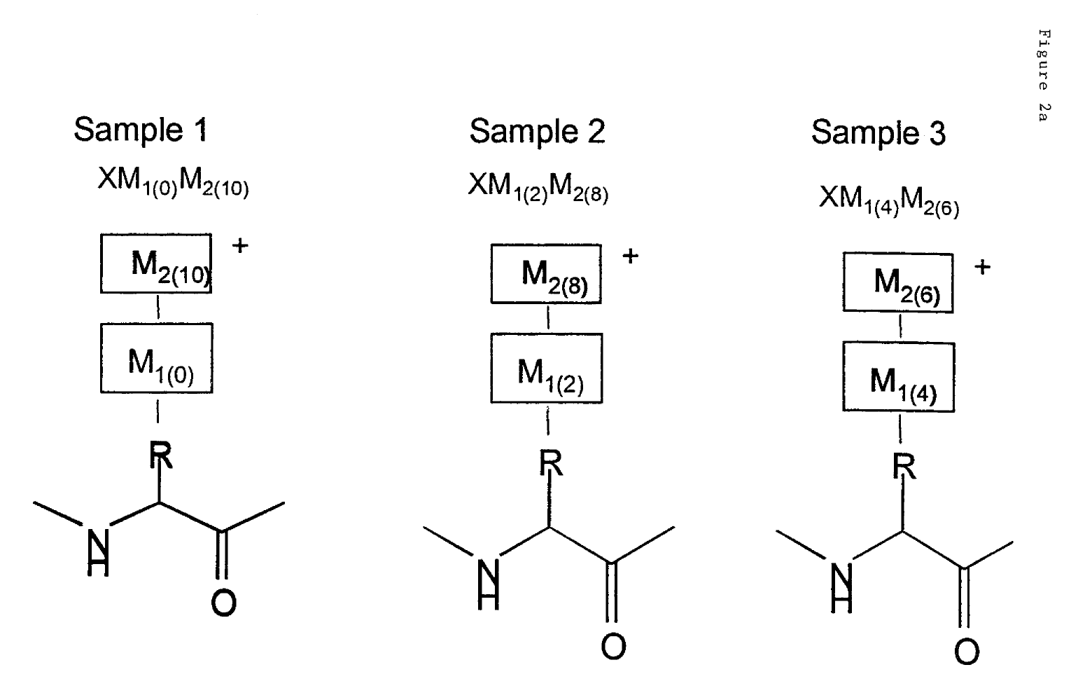

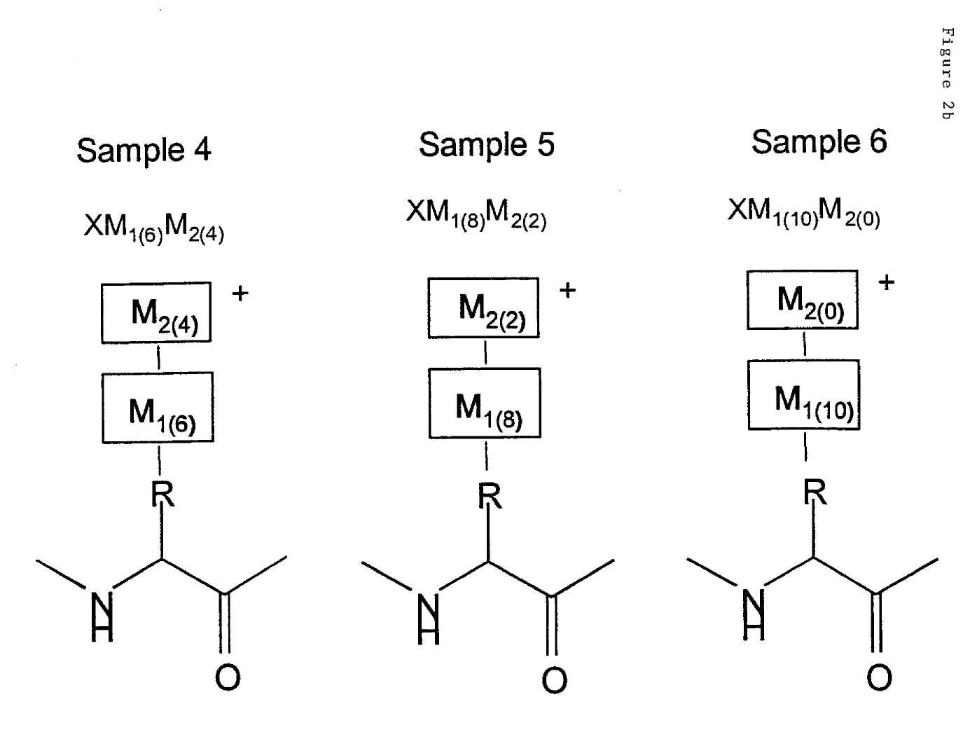

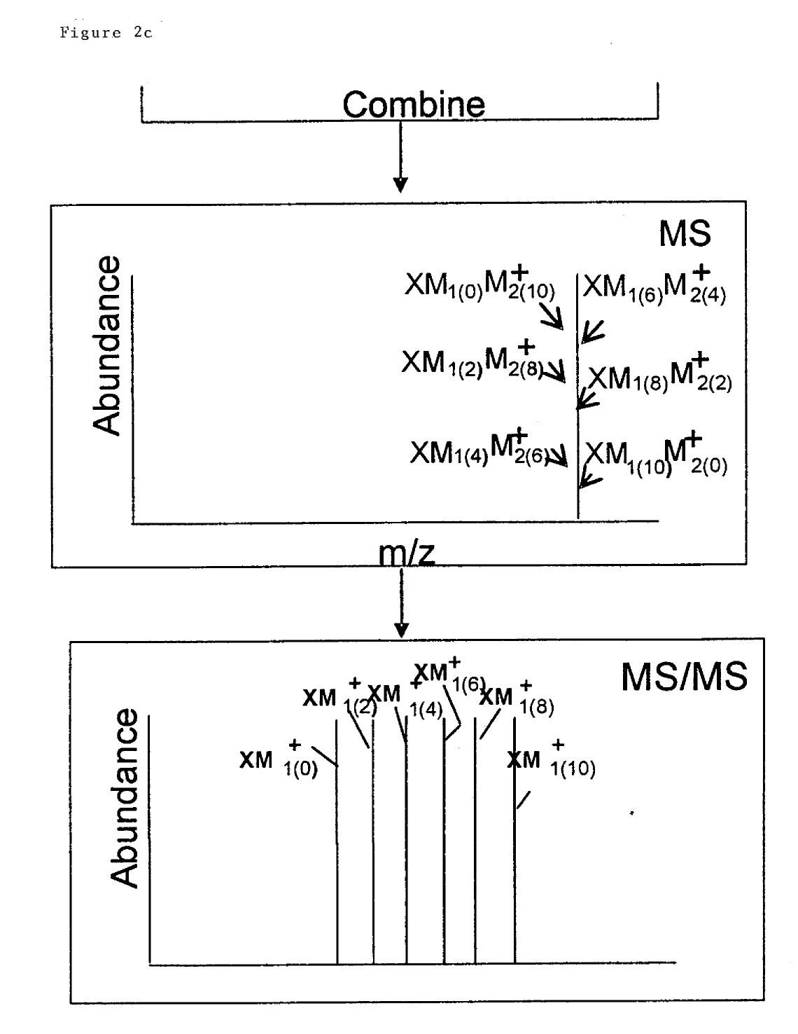

The reagents of the present invention may be used for the ‘multiplexed’ quantification of protein abundances observed between different samples in a single product ion scan mode MS/MS experiment, using the ‘modular’ fixed charge stable isotope labelling approach described below (shown in FIG. 1 for reaction with alkylation reagents XM₁M₂′⁺ and XM₁′M₂⁺). Here, derivatization of a first ‘normal’ sample is carried out using an isotopically distinct labelled alkylation reagent XM₁M₂′⁺, where the M₁ module contains only naturally abundant isotopes and where the M₂′⁺ ‘module’ is isotopically enriched (for example with ²H, ¹³C, ¹⁵N or ¹⁸O), preferably giving an increase of up to twelve mass units compared to an M₂⁺ module containing only naturally abundant isotopes. Simultaneously, derivatization of multiple ‘diseased’ samples may be carried out using (i) the isotopically distinct labelled alkylation reagent XM₁′M₂⁺, where the M₁′ ‘module’ is isotopically enriched (for example with ²H, ¹³C, ¹⁵N or ¹⁸O), preferably giving an increase of up to twelve mass units compared to an M₁ module containing only naturally abundant isotopes while the M₂⁺ module contains only naturally abundant isotopes, and (ii) the isotopically distinct labelled alkylation reagents XM₁′M₂′⁺, where the M₁′ ‘modules’ contain an increasing number of isotopically enriched labels (in increments of one, two, three or four mass units, thereby allowing multiplexed analysis of 12, 6, 4 or 3 ‘diseased’ samples', respectively) compared to that used in the ‘normal’ sample, while the M₂′⁺ ‘modules’ contain an equally decreasing number of isotopically enriched labels (for example ²Hₙ, ¹³Cₙ, ¹⁵Nₙ or ¹⁸Oₙ) compared to that used in the ‘normal’ sample. The masses of each of the alkylation reagents employed for labelling both ‘normal’ and ‘diseased’ samples are then identical, such that the mass difference between ‘normal’ and ‘diseased’ derivatized samples is zero.

The samples are then combined and subjected to tandem mass spectrometry. Quantitative analysis of the relative peptide concentrations between the ‘normal’ and ‘diseased’ samples may then be achieved in a single product ion scan mode MS/MS experiment by measurement of the abundances of the isotopically distinct M₁ and M₁containing product ions formed by neutral loss of M₂′ or M₂, or by measurement of the abundances of the isotopically distinct M₂′ and M₂ product ions formed by charged loss of M₂′ and M₂, respectively, via directed fragmentation occurring at the bond between the M₁ and M₂⁺ modules.

### Example 4

In this example, six different protein samples are each derivatized with one of six versions (0/10, 2/8, 4/6, 6/4, 8/2, 10/0) of the 11 preferred thiol reactive reagents described above. These reagents will label the amino acid cysteine. The same procedure as in Example 3 is followed.

In MS a single peak representing 6 different samples is obtained. In MS/MS these six samples are separated into 6 different peaks using lower CID energy. The peaks may be assigned to the respective sample and the peaks may be identified using higher CID energy, quantified and relatively quantified in relation to each other.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

1. A fixed charge reagent of formula XM₁M₂⁺, or a salt thereof, 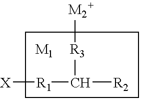
wherein,
X is a thiol, a thiol reactive group, an amino reactive group, a guanidino reactive group, or a reactive group specific for the C₂-indole position of the side chain of Trp or a phosphate reactive group;
M₁ is —R₁CH(R₃)R₂, where R₁ is selected from —(CH₂)ₙ—, —Y— or —(CH₂)ₙY— or is absent; R₂ is —YCH₂COC₆H₅, or when X is a phosphate reactive group R₂ is selected from —(CH₂)ₙH, —C₆H₅, —CH₂C₆H₅, —NH₂, —YH, —Y(CH₂)ₙH, —YC₆H₅ or —YCH₂C₆H₅; and R₃ is —(CH₂)ₙ—, and is optionally isotopically encoded by incorporation of one or more of ²H, ¹³C, ¹⁵N or ¹⁸O in which case M₁ is referred to as M₁′.
n is from 1 to 3 inclusive;
Y is selected from CONH, NHCO, and COO; and
M₂⁺ is attached to the R′₁₃ group of M₁′ and is selected from the group consisting of a tertiary alkyl or aryl sulfonium ion, —S⁺CH₃R″, where R″ is selected from —CH₂COC₆H₅, —CH₂COC₆H₅CH₃, and —CH₂COC₆H₅CH₂CH₃, or a quaternary alkyl or aryl ammonium ion —N⁺(R′″)₃, or a quaternary alkyl or aryl phosphonium ion —P⁺(R′″)₃, where R′″₃ is —(CH₂)ₙH (where n=1 to 3), —C₆H₅, —CH₂C₆H₅, —CH₂COC₆H₅ and wherein M₂⁺ optionally is isotopically encoded by incorporation of one or more ²H, ¹³C, ¹⁵N or ¹⁸O and is in that case referred to as M₂′⁺.

2. The reagent of claim 1, wherein the thiol reactive X group is selected from the group consisting of a halide, a disulfide exchange group, a vinyl group or a N-methyl maleimide group.

3. The reagent of claim 2, wherein X is a halide selected from —Cl, —Br and —I.

4. The reagent of claim 2, wherein X is a disulfide exchange group selected from —S—S—R′ where R′ is —C₆H₅, 3-carboxyl-4-nitrophenyl, 2,4-dinitrophenyl, 4-nitrophenyl, 2-nitrophenyl, 2-pyridyl, 5-nitropyridyl, 3-nitropyridyl, methanesulfonyl.

5. The reagent of claim 1, wherein the amino reactive X group is selected from the group consisting of an acid anhydride, an active ester, an acid halide, a sulfonylhalide, a substituted O-methyl isourea, an isocyanate or an isothiocyanante.

6. The reagent of claim 1, wherein the guanidino reactive X group is selected from the group consisting of a substituted 2,3-butanedione, a substituted 2,4-pentanedione, a substituted glyoxal, or a substituted phenylglyoxal.

7. The reagent of claim 1, wherein the reactive X group specific to the C2-indole position of the side chain of tryptophan or tryptophan containing proteins or peptides is selected from a halide, sulfenylhalide or a dimethyl sulfonium ion.

8. The reagent of claim 1, wherein the phosphate reactive X group is selected from X-groups specific to phosphorylated amino acids.

9. The reagent of claim 1 comprising the following formula

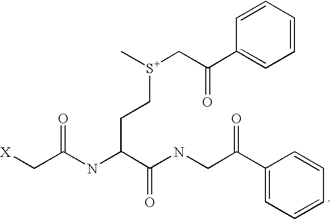

10. The reagent of claim 9, wherein X is selected from a thiol reactive group; an amino reactive group; or a phosphate reactive group.

11. A reagent kit for MS analysis, comprising one or more of the reagents XM₁′M₂⁺, XM₁M₂⁺′ and XM₁′M₂⁺′ of claim 9.

12. The kit of claim 11, comprising one or more amino acids, peptides or proteins derivatized with XM₁′M₂⁺, XM₁M₂⁺′ and XM₁′M₂⁺′ for use as standards.

13. The kit of claim 11, comprising two or more of the following reagents

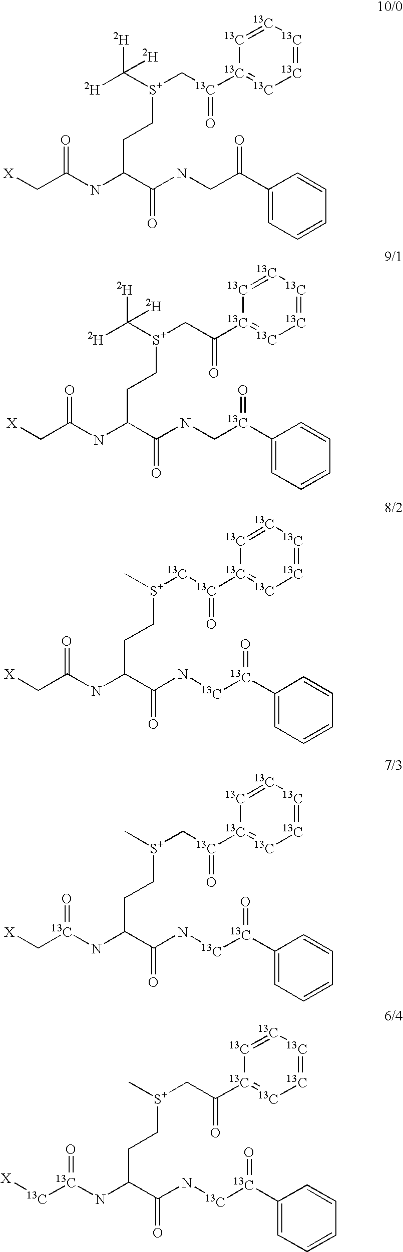

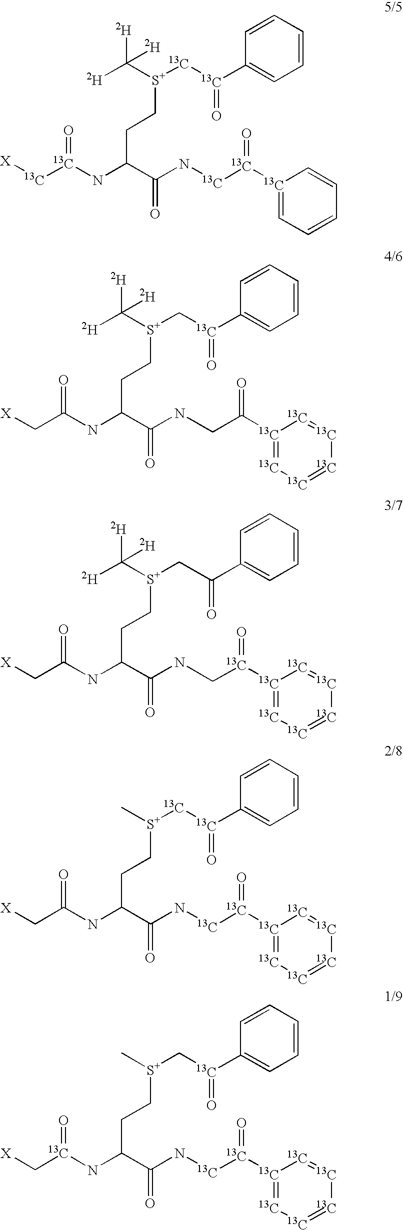

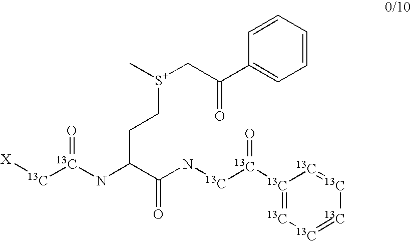

14. The kit of claim 11, comprising two or more of the following reagents

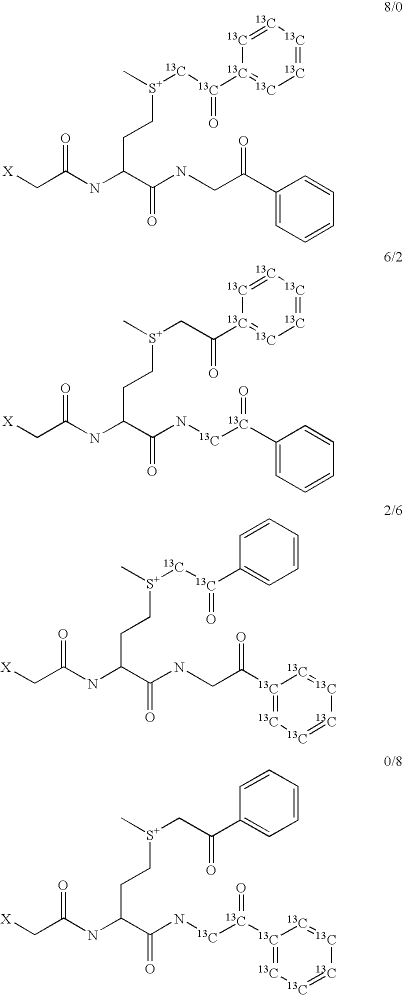

15. The kit of claim 13, wherein X is a halide selected from Br and I.

16. The kit of claim 13, wherein X is an NHS-ester.

17. A method for quantitative analysis of amino acids, peptides or proteins, wherein one or more of the reagents of claim 9 is used, the method comprising:
(1) providing a mixture of amino acids, peptides or proteins containing at least one selected amino acid, peptide or protein, or peptide or protein comprising at least one residue of the selected amino acid, derivatized to contain a fixed-charge using compounds of formula XM₁′M₂⁺, XM₁M₂′⁺ and XM₁′M₂′⁺, or salts thereof, as described above;
(2) passing the mixture of amino acids, peptides or proteins containing at least one derivatized amino acid or derivatized amino acid residue containing peptide or protein, through a first mass resolving spectrometer to select precursor protein or peptide ions having a first mass-to-charge ratio;
(3) subjecting the precursor ions of the first mass-to-charge ratio to dissociation to form a product ion having a second mass-to-charge ratio that is characteristic of the loss of M₂ or M₂′ at the site of the fixed-charge; and
(4) detecting the product ions having the second mass-to-charge ratio.

18. The method of claim 17, wherein the amino acid, peptide or protein contains an N-terminal amino group, a cysteine, a homocysteine, a lysine, an arginine, a homoarginine, a tryptophan, a dehydroalanine or a dehydroamino-2-butyric acid, or a phosphate.

19-21. (canceled)

22. The kit of claim 14, wherein X is a halide selected from Br and I.

23. The kit of claim 14, wherein X is an NHS-ester.
